(19) United States
(12) Patent Application Publication
(10) Pub. No.: US 2005/0191362 A1
(43) Pub. Date: Sep. 1, 2005

(54) Method of preparing microcapsules encapsulating a phase transition material

(76) Inventor: Yen-Hsi Lin, Taipei City (TW)

Correspondence Address: KNOBBE MARTENS OLSON & BEAR LLP, 2040 MAIN STREET, FOURTEENTH FLOOR, IRVINE, CA, 92614, US

(21) Appl. No.: 10/969,661

(22) Filed: Oct. 20, 2004

(30) Foreign Application Priority Data
Mar. 1, 2004 (TW) 93105315

(51) Int. Cl. A61K 9/16; A61K 9/50

(52) U.S. Cl. 424/490; 264/4.1

(57) ABSTRACT

A phase transition material, an acrylic monomer and a free-radical initiator are dissolved in an organic solvent to form an oil-phase solution. The acrylic monomer, having bi-functional and/or tri-functional groups, undergoes free radical polymerization to form the microcapsules shells encapsulating the phase transition material. A surfactant is dissolved in water to form a water-phase solution, and the HLB value of the surfactant is eight to twelve. The oil-phase solution and the water-phase solution are mixed and stirred to form an emulsion solution. The emulsion solution is then heated stepwise to form the microcapsules encapsulating the phase transition material.

### RELATED APPLICATIONS

The present application is based on, and claims priority from, Taiwan Application Serial Number 93105315, filed on Mar. 1, 2004, the disclosure of which is hereby incorporated by reference herein in its entirety.

### BACKGROUND OF THE INVENTION

1. Field of Invention

The present invention relates to a method of preparing microcapsules encapsulating a phase transition material. More particularly, the present invention relates to a method of preparing microcapsules encapsulating a phase transition material by using an acrylic monomer having bi- and/or tri-functional groups to form microcapsule shells via a free radical polymerization.

2. Description of Related Art

A phase transition material is a material that can transition between solid phase and liquid phase in a certain temperature range. During the phase transition, a lot of latent heat is released or absorbed. A common phase transition material is paraffinic hydrocarbons (CₙH₂ₙ₊₂). The main feature of the phase transition material is that a system temperature can be held at a certain temperature when the phase transition material absorbs or releases a lot of latent heat. Hence, a common application of phase transition materials is the manufacture of thermal insulation textiles.

However, if the phase transition material coating on the textile directly is used to make thermal insulation textiles, the phase transition material leaks from the textile when it undergoes the phase transition from solid to liquid. Therefore, at least a thin film is needed to wrap the phase transition material, and the phase transition material is then implanted or coated on the textiles to resolve the above problem. One solution to the above problem is microcapsule technology.

For example, U.S. Pat. No. 6,200,681 utilizes free radical polymerization to form microcapsules, which encapsulate phase transition materials. The main feature is using from 30 to 100 wt. % of one or more C1-C24 alkyl esters of acrylic and/or methacrylic acid, which has a mono-functional group, from 0 to 80 wt. % of bi- or poly-functional monomer, and from 0 to 40 wt. % of other monomers to form shells of the microcapsules. A phase transition material having a phase transition temperature of −20° C. to 120° C. is used as cores of the microcapsules. The phase transition material is alkyl or aromatic hydrocarbon compounds, saturated or unsaturated C6-C30 fatty acids, fatty alcohols, C6-C30 fatty amines, esters such as C1-C10 alkyl esters of fatty acids, natural and synthetic waxes, or halogenated hydrocarbons. The C1-C10 alkyl esters of fatty acids can be propyl palmitate, methyl stearate or methyl palmitate and, preferably, their eutectic mixtures, or methyl cinnamate.

In U.S. Pat. No. 5,456,852, an in-situ polymerization of melamine-formaldehyde resins and suitable surfactant is used to form microcapsule shells. The heat-storing material includes a phase transition material and a high-melting compound. The phase transition material includes straight-chain aliphatic hydrocarbons having 10 or more carbon atoms, alkyl myristates, alkyl palmitate, alkyl stearate, and mixtures thereof. The high-melting compound includes cetyl alcohol, stearyl alcohol, eicosanol, myristic acid, palmitic acid, behenic acid, stearic acid amide, ethylenebisoleic acid amide, methylolbehenic acid amide and N-phenyl-N′-stearylurea, and its melting point is 20° C. to 110° C.

In U.S. Pat. No. 4,708,812, prepolymer of isocyanate-terminated polyurethane is synthesized by prepolymerization-interfacial condensation polymerization and then added to a mixture solution of a phase transition material and a surfactant. Next, an aqueous solution of polyamine is added to the mixture solution and then agitated by an agitator to form an emulsion. Polyurethane-polyurea is synthesized by interfacial condensation polymerization in the emulsion to form microcapsules shells encapsulating phase transition materials. The phase transition materials consist of a crystalline polymer, naphthalene, salt hydrates and a crystalline paraffin, such as Paravan 450, Slack Wax 3645, or Slack Wax 3663.

### SUMMARY OF THE INVENTION

In one aspect, the present invention provides a method of preparing microcapsules encapsulating phase transition material. Hence, shell density of the microcapsules is elevated to solve the problem of phase transition material leakage.

In another aspect, the present invention provides a method of preparing microcapsules encapsulating phase transition material. In this method, rancid C1-C24 alkyl esters of acrylic and methacrylic acid are not used to decrease the harm to the human body.

In accordance with the foregoing and other aspects of the present invention, this invention provides a method of preparing microcapsules encapsulating a phase transition material. A solid-liquid phase transition material, an acrylic monomer having bi- and/or tri-functional groups and a free radical initiator are dissolved in an organic solvent to from an oil-phase solution. A surfactant is dissolved in water to form a water-phase solution, and a hydrophilic-lipophilic balance (HLB) value of the surfactant is about 8 to about 12. The oil-phase solution and the water-phase solution are mixed and then agitated to form an emulsion. Then, the emulsion is heated at a gradient temperature to form microcapsules encapsulating the solid-liquid phase transition material.

In the foregoing, the acrylic monomer having bi-functional groups is preferably 1,6-hexanediol diacrylate, dipropylene glycol diacrylate, tripropylene glycol diacrylate, polyethylene glycol(200-400-600)diacrylate, neopentyl glycol diacrylate, ethoxylated bisphenol-a diacrylate, 2-butyl-2-ethyl-1,3-propanediol diacrylate, dipropylene glycol dimethacrylate, ethoxylated bisphenol-a diemthacrylate, diethylene glycol dimethacrylate, or a combination thereof. The acrylic monomer having bi-functional groups is more preferably 1,6-hexanediol diacrylate or dipropylene glycol diacrylat.

The acrylic monomer having tri-functional groups is preferably trimethylolpropane triacrylate, trimethylolpropane trimethacrylate, ethoxylated trimethylolpropane triacrylate, propoxylated trimethylolpropane triacrylate, propoxylated glyceryl triacrylate, ethoxylated trimethylolpropane trimethacrylate or a combination thereof. The acrylic monomer having tri-functional groups is more preferably ethoxylated trimethylolpropane triacrylate or trimethylolpropane triacrylate.

As embodied and broadly described herein, the invention only uses acrylic monomer having bi- and/or tri-functional groups to form microcapsule shells via free radical polymerization. Since the dense network structure formed by the polymerization of acrylic monomer has bi- and/or tri-functional groups, the liquid phase transition material can be completely encapsulated without any leakage. Furthermore, C1-C24 alkyl esters of acrylic and methacrylic acid are not used; the rancid smell and harm to operators can therefore be decreased.

It is to be understood that both the foregoing general description and the following detailed description are by examples, and are intended to provide further explanation of the invention as claimed.

### DESCRIPTION OF THE PREFERRED EMBODIMENTS

This invention provides a method of preparing microcapsules encapsulating phase transition material to form dense microcapsule shells.

This invention reveals that if only acrylic monomers having bi- and/or tri-functional groups are used to form microcapsule shells, a hermetic network polymer is formed by the acrylic monomers having bi- and/or tri-functional groups to seal the phase transition material. Hence, the phase transition material does not leak when the phase transition material is in the liquid phase. The acrylic monomer having bi-functional groups over the acrylic monomer having tri-functional groups is about 3:2 to 0:1. Moreover, the preparation method of this invention is simpler than that of the U.S. Pat. No. 6,200,681 using the C1-C24 alkyl esters of acrylic and/or methacrylic acid having mono-functional group.

Therefore, according to a preferred embodiment of this invention, cores of microcapsules include one or more phase transition materials, which undergo phase transition between solid and liquid phase at a temperature of −20° C. to 80° C. The shells of the microcapsules are formed by acrylic monomer having bi- and/or tri-functional groups via free radical polymerization.

The method of preparing microcapsules encapsulating phase transition materials includes the following steps. A surfactant is dissolved in water to form an aqueous solution. The surfactant concentration is about 1.5-2.5 wt. %. A phase transition material, acrylic monomers having bi- and/or tri-functional groups, a free radical initiator and an organic solvent are mixed to form an organic solution. The aqueous solution and the organic solution are mixed and emulsified by a high-speed homogeneous mixer for 2-5 minutes. The agitation speed of the homogeneous mixer is about 2500-6000 rpm. The emulsion is heated at a gradient temperature, about 50-70° C., for 4-6 hours. That is, the emulsion is heated at least at two constant temperatures for 1-3 hours, respectively. Consequently, an aqueous solution of microcapsules encapsulating phase transition materials is obtained, and the diameters of the microcapsules are less than several microns. The aqueous solution of the microcapsules can be either used directly or freeze-dried to form powder for later use.

The acrylic monomer having bi-functional groups is preferably 1,6-hexanediol diacrylate, dipropylene glycol diacrylate, tripropylene glycol diacrylate, polyethylene glycol(200-400-600)diacrylate, neopentyl glycol diacrylate, ethoxylated bisphenol-a diacrylate, 2-butyl-2-ethyl-1,3-propanediol diacrylate, dipropylene glycol dimethacrylate, ethoxylated bisphenol-a diemthacrylate, diethylene glycol dimethacrylate, or a combination thereof. The acrylic monomer having bi-functional groups is more preferably 1,6-hexanediol diacrylate or dipropylene glycol diacrylat.

The acrylic monomer having tri-functional groups is preferably trimethylolpropane triacrylate, trimethylolpropane trimethacrylate, ethoxylated trimethylolpropane triacrylate, propoxylated trimethylolpropane triacrylate, propoxylated glyceryl triacrylate, ethoxylated trimethylolpropane trimethacrylate or a combination thereof. The acrylic monomer having tri-functional groups is more preferably ethoxylated trimethylolpropane triacrylate or trimethylolpropane triacrylate.

The phase transition temperature of the phase transition material is about −20° C. to 80° C. The phase transition material is preferably an ester of a carboxylic acid, an aliphatic or aromatic hydrocarbon compound, a saturated or unsaturated C6-C30 aliphatic acid, an aliphatic alcohol, a C6-C30 aliphatic amine, an ester, a natural wax, a synthetic wax, a halo-hydrocarbon compound, or a combination thereof. The carboxylic acid of the ester is, for example, formic acid, acetic acid, or propionic acid, and the alcohol of the ester is, for example, a saturated aliphatic alcohol having 10 to 28 carbons.

The hydrophilic-lipophilic balance (HLB) value of the surfactant is preferably about 8-12. The surfactant is preferably polyoxyethylene stearyl cetyl ether, sorbitan laurate, polyoxyethylene sorbitan oleate, polyoxyethylene sorbitan trioleate, a nonionic anionic surfactant, or a mixture of any two or three surfactants described above.

The free radical initiator is preferably a peroxide, which is common in commercial use. The peroxide is, for example, a tert-butyl hydroperoxide, di-tert-butyl peroxide, or benzoyl peroxide.

Several working examples are provided below to further explain the principles of this invention.

### EXAMPLE 1

| Phase | Compositions | Weight (g) |
|---|---|---|
| Water | Sorbitan laurate | 5.3 |
| | Water | 302 |
| Oil | 1,6-hexanediol diacrylate | 9.1 |
| | Ethoxylated trimethylolpropane triacrylate | 6.2 |
| | Stearyl acetate | 101 |
| | Benzoyl peroxide | 0.51 |
| | Acetyl acetate | 5 mL |

5.3 g of sorbitan laurate was added to 302 g of water. The mixture was then heated to dissolve the sorbitan laurate in the water to form a water phase solution (a continuous phase). In addition, 9.1 g of 1,6-hexanediol diacrylate, 6.2 g of ethoxylated trimethylolpropane triacrylate, 101 g of Stearyl acetate, 0.51 g of benzoyl peroxide, and 5 mL of acetyl acetate were mixed and agitated to form an oil phase solution (a dispersed phase). Then, the water phase solution and the oil phase solution were mixed and emulsified by agitating at 5000 rpm for 3 minutes. After heating the mixture solution at 60° C. for 2 hours and then at 70° C. for 3 hours, microcapsules having diameters less than 2.6 μm were obtained. A powder of microcapsules encapsulating phase transition material was obtained by freeze-drying.

### EXAMPLE 2

| Phase | Compositions | Weight (g) |
|---|---|---|
| Water | Polyoxyethylene sorbitan oleate | 5.0 |
| | Water | 303 |
| Oil | 1,6-hexanediol diacrylate | 9.0 |
| | Ethoxylated trimethylolpropane triacrylate | 6.0 |
| | Stearyl acetate | 101 |
| | Benzoyl peroxide | 0.498 |
| | Acetyl acetate | 5 mL |

5.0 g of polyoxyethylene sorbitan oleate was added to 303 g of water. The mixture was then heated to dissolve the polyoxyethylene sorbitan oleate in the water to form a water phase solution (a continuous phase). In addition, 9.0 g of 1,6-hexanediol diacrylate, 6.0 g of ethoxylated trimethylolpropane triacrylate, 101 g of Stearyl acetate, 0.498 g of benzoyl peroxide, and 5 mL of acetyl acetate were mixed and agitated to form an oil phase solution (a dispersed phase). Then, the water phase solution and the oil phase solution were mixed and emulsified by agitating at 5500 rpm for 3 minutes. After heating the mixture solution at 55° C. for 1 hour, at 60° C. for 2 hours and then at 70° C. for 2 hours, microcapsules having diameters less than 1.8 μm were obtained. A powder of microcapsules encapsulating phase transition material was obtained by freeze-drying.

### EXAMPLE 3

| Phase | Compositions | Weight (g) |
|---|---|---|
| Water | Sorbitan laurate | 3.4 |
| | Polyoxyethylene sorbitan oleate | 3.4 |
| | Water | 301 |
| Oil | 1,6-hexanediol diacrylate | 9.1 |
| | Ethoxylated trimethylolpropane triacrylate | 6.2 |
| | Stearyl acetate | 100 |
| | Benzoyl peroxide | 0.51 |
| | Acetyl acetate | 5 mL |

3.4 g of sorbitan laurate and 3.4 g of polyoxyethylene sorbitan oleate were added to 301 g of water. The mixture was then heated to dissolve the sorbitan laurate and polyoxyethylene sorbitan oleate in the water to form a water phase solution (a continuous phase). In addition, 9.1 g of 1,6-hexanediol diacrylate, 6.2 g of ethoxylated trimethylolpropane triacrylate, 100 g of Stearyl acetate, 0.51 g of benzoyl peroxide, and 5 mL of acetyl acetate were mixed and agitated to form an oil phase solution (a dispersed phase). Then, the water phase solution and the oil phase solution were mixed and emulsified by agitating at 3000 rpm for 3 minutes. After heating the mixture solution at 60° C. for 2 hours and then at 70° C. for 3 hours, microcapsules having diameters less than 4.5 μm were obtained. A powder of microcapsules encapsulating phase transition material was obtained by freeze-drying.

### EXAMPLE 4

| Phase | Compositions | Weight (g) |
|---|---|---|
| Water | Sorbitan laurate | 3.0 |
| | Polyoxyethylene stearyl cetyl ether | 3.0 |
| | Water | 300 |
| Oil | 1,6-hexanediol diacrylate | 9.0 |
| | Ethoxylated trimethylolpropane triacrylate | 6.0 |
| | Stearyl acetate | 100 |
| | Benzoyl peroxide | 0.5 |
| | Acetyl acetate | 5 mL |

3.0 g of sorbitan laurate and 3.0 g of polyoxyethylene stearyl cetyl ether were added to 300 g of water. The mixture was then heated to dissolve the sorbitan laurate and polyoxyethylene stearyl cetyl ether in the water to form a water phase solution (a continuous phase). In addition, 9.0 g of 1,6-hexanediol diacrylate, 6.0 g of ethoxylated trimethylolpropane triacrylate, 100 g of Stearyl acetate, 0.50 g of benzoyl peroxide, and 5 mL of acetyl acetate were mixed and agitated to form an oil phase solution (a dispersed phase). Then, the water phase solution and the oil phase solution were mixed and emulsified by agitating at 3000 rpm for 3 minutes. After heating the mixture solution at 55° C. for 1 hour, at 60° C. for 4 hours and then at 70° C. for 1 hour, microcapsules having diameters less than 4.1 μm were obtained. A powder of microcapsules encapsulating phase transition material was obtained by freeze-drying.

### EXAMPLE 5

| Phase | Compositions | Weight (g) |
|---|---|---|
| Water | Sorbitan laurate | 3.4 |
| | Polyoxyethylene stearyl cetyl ether | 3.4 |
| | Water | 400 |
| Oil | 1,6-hexanediol diacrylate | 12.0 |
| | Ethoxylated trimethylolpropane triacrylate | 8.1 |
| | Stearyl acetate | 26 |
| | Stearyl propionate | 76 |
| | Benzoyl peroxide | 0.65 |
| | Acetyl acetate | 6 mL |

3.4 g of sorbitan laurate and 3.4 g of polyoxyethylene stearyl cetyl ether were added to 400 g of water. The mixture was then heated to dissolve the sorbitan laurate and polyoxyethylene stearyl cetyl ether in the water to form a water phase solution (a continuous phase). In addition, 12.0 g of 1,6-hexanediol diacrylate, 8.1 g of ethoxylated trimethylolpropane triacrylate, 26 g of stearyl acetate, 76 g of stearyl propionate, 0.65 g of benzoyl peroxide, and 6 mL of acetyl acetate were mixed and agitated to form an oil phase solution (a dispersed phase). Then, the water phase solution and the oil phase solution were mixed and emulsified by agitating at 5500 rpm for 3 minutes. After heating the mixture solution at 60° C. for 3 hours and then at 70° C. for 2 hours, microcapsules having diameters less than 1.5 μm were obtained. A powder of microcapsules encapsulating phase transition material was obtained by freeze-drying.

### EXAMPLE 5

| Phase | Compositions | Weight (g) |
|---|---|---|
| Water | Sorbitan laurate | 3.4 |
| | Polyoxyethylene stearyl cetyl ether | 3.4 |
| | Water | 415 |
| Oil | Trimethylolpropane triacrylate | 15 |
| | Stearyl acetate | 26 |
| | Stearyl propionate | 77 |
| | Benzoyl peroxide | 0.6 |
| | Acetyl acetate | 5 mL |

3.4 g of sorbitan laurate and 3.4 g of polyoxyethylene stearyl cetyl ether were added to 415 g of water. The mixture was then heated to dissolve the sorbitan laurate and polyoxyethylene stearyl cetyl ether in the water to form a water phase solution (a continuous phase). In addition, 15 g of trimethylpropane triacrylate, 26 g of stearyl acetate, 76 g of stearyl propionate, 0.6 g of benzoyl peroxide, and 5 mL of acetyl acetate were mixed and agitated to form an oil phase solution (a dispersed phase). Then, the water phase solution and the oil phase solution were mixed and emulsified by agitating at 3500 rpm for 5 minutes. After heating the mixture solution at 60° C. for 3 hours and then at 70° C. for 2 hours, microcapsules having diameters less than 2.0 μm were obtained. A powder of microcapsules encapsulating phase transition material was obtained by freeze-drying.

In light of the foregoing, the invention only uses acrylic monomer having bi- and/or tri-functional groups to form microcapsule shells via free radical polymerization. Since the dense network structure formed by the polymerization of acrylic monomer having bi- and/or tri-functional groups, the liquid phase transition material can be completely encapsulated without any leakage. Furthermore, C1-C24 alkyl esters of acrylic and methacrylic acid are not used; the rancid smell and harm to operators can therefore be decreased.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

1. A method of preparing microcapsules encapsulating a phase transition material, comprising:
dissolving a solid-liquid phase transition material, an acrylic monomer and a free radical initiator in an organic solvent to from an oil-phase solution, wherein the acrylic monomer, having bi- and/or tri-functional groups, is used to form shells via a free radical polymerization;
dissolving a surfactant in water to form a water-phase solution, a HLB value of the surfactant being about 8 to about 12;
mixing the oil-phase solution and the water-phase solution to form a mixture solution;
agitating the mixture solution to form an emulsion; and
heating the emulsion at a gradient temperature to form microcapsules encapsulating the solid-liquid phase transition material.

2. The method of claim 1, wherein a phase transition temperature of the solid-liquid phase transition material is about −20° C. to about 80° C.

3. The method of claim 1, wherein the solid-liquid phase transition material comprises an ester of a carboxylic acid.

4. The method of claim 3, wherein the carboxylic acid of the ester comprises formic acid, acetic acid, or propionic acid, and the alcohol of the ester is a saturated aliphatic alcohol having 10 to 28 carbons.

5. The method of claim 1, wherein the solid-liquid phase transition material is selected from a group consisting of an ester of a carboxylic acid, an aliphatic or aromatic hydrocarbon compound, a saturated or unsaturated C6-C30 aliphatic acid, an aliphatic alcohol, a C6-C30 aliphatic amine, an ester, a natural wax, a synthetic wax, a halo-hydrocarbon compound and a combination thereof.

6. The method of claim 1, wherein the acrylic monomer having bi-functional groups is selected from a group consisting of 1,6-hexanediol diacrylate, dipropylene glycol diacrylate, tripropylene glycol diacrylate, polyethylene glycol(200-400-600)diacrylate, neopentyl glycol diacrylate, ethoxylated bisphenol-a diacrylate, 2-butyl-2-ethyl-1,3-propanediol diacrylate, dipropylene glycol dimethacrylate, ethoxylated bisphenol-a diemthacrylate, diethylene glycol dimethacrylate, and a combination thereof.

7. The method of claim 1, wherein the acrylic monomer having bi-functional groups comprises 1,6-hexanediol diacrylate.

8. The method of claim 1, wherein the acrylic monomer having bi-functional groups comprises dipropylene glycol diacrylate.

9. The method of claim 1, wherein the acrylic monomer having tri-functional groups is selected from a group consisting of trimethylolpropane triacrylate, trimethylolpropane trimethacrylate, ethoxylated trimethylolpropane triacrylate, propoxylated trimethylolpropane triacrylate, propoxylated glyceryl triacrylate, ethoxylated trimethylolpropane trimethacrylate and a combination thereof.

10. The method of claim 1, wherein the acrylic monomer having tri-functional groups comprises ethoxylated trimethylolpropane triacrylate.

11. The method of claim 1, wherein the acrylic monomer having tri-functional groups comprises trimethylolpropane triacrylate.

12. The method of claim 1, wherein the acrylic monomer comprises a molar ratio of the acrylic monomer having bi-functional groups to the acrylic monomer having tri-functional groups is about 3:2 to 0:1.

13. The method of claim 1, wherein the free radical initiator comprises a peroxide.

14. The method of claim 13, wherein the peroxide comprises a tert-butyl hydroperoxide, di-tert-butyl peroxide, or benzoyl peroxide.

15. The method of claim 1, wherein the surfactant is selected from a group consisting of polyoxyethylene stearyl cetyl ether, sorbitan laurate, polyoxyethylene sorbitan oleate, polyoxyethylene sorbitan trioleate, a nonionic anionic surfactant and a combination thereof.

16. The method of claim 1, wherein a temperature of the gradient heating is about 50-70° C.

17. The method of claim 1, wherein a heating time of the gradient heating is about 4-6 hours.

18. The method of claim 1, wherein the organic solvent comprises acetyl acetate.

19. A composition of an emulsion for preparing microcapsules encapsulating a phase transition material, comprising:
a solid-liquid phase transition material dissolved in an organic phase of the emulsion;
an acrylic monomer dissolved in the organic phase of the emulsion, the acrylic monomer having bi- and/or tri-functional groups;
a free radical initiator dissolved in the organic phase of the emulsion; and
a surfactant dissolved in a water phase of the emulsion, a hydrophilic-lipophilic balance value of the surfactant being 8-12.

20. The composition of claim 19, wherein a phase transition temperature of the solid-liquid phase transition material is about −20 to about 80° C.

21. The composition of claim 19, wherein the solid-liquid phase transition material comprises an ester of a carboxylic acid.

22. The composition of claim 19, wherein the carboxylic acid of the ester comprises formic acid, acetic acid, or propionic acid, and the alcohol of the ester is a saturated aliphatic alcohol having a carbon number between about 10 and about 28.

23. The composition of claim 19, wherein the solid-liquid phase transition material is selected from a group consisting of an ester of a carboxylic acid, an aliphatic or aromatic hydrocarbon compound, a saturated or unsaturated C6-C30 aliphatic acid, an aliphatic alcohol, a C6-C30 aliphatic amine, an ester, a natural wax, a synthetic wax, a halo-hydrocarbon compound and a combination thereof.

24. The composition of claim 19, wherein the acrylic monomer having bi-functional groups is selected from a group consisting of 1,6-hexanediol diacrylate, dipropylene glycol diacrylate, tripropylene glycol diacrylate, polyethylene glycol(200-400-600)diacrylate, neopentyl glycol diacrylate, ethoxylated bisphenol-a diacrylate, 2-butyl-2-ethyl-1,3-propanediol diacrylate, dipropylene glycol dimethacrylate, ethoxylated bisphenol-a diemthacrylate, diethylene glycol dimethacrylate, and a combination thereof.

25. The composition of claim 19, wherein the acrylic monomer having bi-functional groups comprises 1,6-hexanediol diacrylate.

26. The composition of claim 19, wherein the acrylic monomer having bi-functional groups comprises dipropylene glycol diacrylate.

27. The composition of claim 19, wherein the acrylic monomer having tri-functional groups is selected from a group consisting of trimethylolpropane triacrylate, trimethylolpropane trimethacrylate, ethoxylated trimethylolpropane triacrylate, propoxylated trimethylolpropane triacrylate, propoxylated glyceryl triacrylate, ethoxylated trimethylolpropane trimethacrylate and a combination thereof.

28. The composition of claim 19, wherein the acrylic monomer having tri-functional groups comprises ethoxylated trimethylolpropane triacrylate.

29. The composition of claim 19, wherein the acrylic monomer having tri-functional groups comprises trimethylolpropane triacrylate.

30. The composition of claim 19, wherein the acrylic monomer comprises a molar ratio of the acrylic monomer having bi-functional groups to the acrylic monomer having tri-functional groups is about 3:2 to 0:1.

31. The composition of claim 19, wherein the free radical initiator comprises a peroxide.

32. The composition of claim 31, wherein the peroxide comprises a tert-butyl hydroperoxide, di-tert-butyl peroxide, or benzoyl peroxide.

33. The composition of claim 19, wherein the surfactant is selected from a group consisting of polyoxyethylene stearyl cetyl ether, sorbitan laurate, polyoxyethylene sorbitan oleate, polyoxyethylene sorbitan trioleate, a nonionic anionic surfactant and a combination thereof.

34. The composition of claim 19, wherein the organic solvent comprises acetyl acetate.
